(19) United States
(12) Patent Application Publication
(10) Pub. No.: US 2024/0318138 A1
(43) Pub. Date: Sep. 26, 2024

(54) METHOD AND DEVICE FOR PREPARING UNIVERSAL PLASMA

(71) Applicant: Universitätsmedizin Greifswald, Greifswald (DE)

(72) Inventors: Andreas Greinacher, Süderholz (DE); Konstanze Aurich, Greifswald (DE)

(21) Appl. No.: 18/030,640

(22) PCT Filed: Oct. 5, 2021
(86) PCT No.: PCT/EP2021/077371

§ 371 Date: Apr. 6, 2023

(30) Foreign Application Priority Data
Oct. 6, 2020 (DE) 10 2020 212 609.7

(51) Int. Cl. C12N 5/078; A61K 35/16; C12M 1/00; C12M 1/02

(52) U.S. Cl. CPC C12N 5/0641; A61K 35/16; C12M 23/58; C12M 27/00; C12M 29/14

(57) ABSTRACT

A first aspect of the present invention relates to a method for preparing blood plasma depleted of anti-A antibodies and/or anti-B antibodies. Furthermore, a second aspect of the invention relates to a blood plasma depleted of anti-A antibodies and/or anti-B antibodies that is obtained or obtainable by the method of the first aspect of the invention. A third aspect of the invention relates to the use of a blood plasma depleted of anti-A antibodies and/or anti-B antibodies that is obtained or obtainable by the method of the first aspect of the invention or of a blood plasma depleted of anti-A antibodies and/or anti-B antibodies of the second aspect of the invention. Furthermore, the invention relates to a blood plasma depleted of anti-A antibodies and/or anti-B antibodies or a blood plasma depleted of anti-A antibodies and/or anti-B antibodies of the second aspect of the invention for use in a therapeutic method or an in-vivo diagnostic method or a surgical method. A fourth aspect of the invention relates to a device for preparing blood plasma depleted of anti-A antibodies and/or anti-B antibodies.

In a first aspect, the present invention relates to a method for preparing blood plasma depleted in anti-A antibodies and/or anti-B antibodies, comprising the steps of: (a) providing blood plasma of blood group A, B or O or a mixture of two or more blood plasmas of blood group A, B and O; (b) providing erythrocytes of blood group A and/or erythrocytes of blood group B and/or erythrocytes of blood group AB; (c) combining the blood plasma according to (a) and the erythrocytes according to (b) to obtain a blood plasma depleted in anti-A antibodies and/or anti-B antibodies compared to the blood plasma according to (a), and a cell sediment; (d) removing the blood plasma depleted in anti-A antibodies and/or anti-B antibodies obtained according to (c) from the cell sediment. Furthermore, in a second aspect, the invention relates to a blood plasma depleted in anti-A antibodies and/or anti-B antibodies obtained or obtainable by the method of the first aspect. According to a third aspect, the invention relates to the use of a blood plasma depleted in anti-A antibodies and/or anti-B antibodies obtained or obtainable by the method of the first aspect or of a blood plasma depleted in anti-A antibodies and/or anti-B antibodies according to the second aspect for preparation of a transfusion solution, for preparation of a coagulation factor concentrate, for preparation of albumin, for preparation of immunoglobulin, for preparation of tissue adhesive, for preparation of a prothrombin complex concentrate (PPSB), for preparation of a cell culture. Furthermore, the invention relates to a blood plasma depleted in anti-A antibodies and/or anti-B antibodies, in particular a blood plasma free of anti-A antibody and/or anti-B antibody, obtained or obtainable by the method of the first aspect described above or a blood plasma depleted in anti-A antibodies and/or anti-B antibodies, in particular a blood plasma free of anti-A antibody and/or anti-B antibody, according to the second aspect for use in a therapeutic method or an in vivo diagnostic method or a surgical method. A fourth aspect of the invention relates to a device for preparing blood plasma depleted in anti-A antibodies and/or anti-B antibodies, comprising: at least one collection container, a connection device which for connection of at least one first container, wherein the first container comprises blood plasma of blood group A, B or O or a mixture of two or more blood plasmas of blood group A, B and O, and at least one second container, wherein the second container comprises erythrocytes of blood group A, erythrocytes of blood group B and/or erythrocytes of blood group AB, to the collection container in such a way that the blood plasma of the first container is combinable with the erythrocytes in the collection container.

Blood plasma must be transfused in an ABO blood group-compatible manner because the anti-A and anti-B antibodies in the plasma will harm the patient if the corresponding antigen is present on the patient's blood cells. Only plasma of blood group AB can be universally transfused to any recipient, since it contains neither A nor B antibodies. However, in the European population, only 4% of donors have blood group AB. A method which removes the antibodies present from plasmas of blood groups A, B and O is therefore of great importance.

Antibody-depleted plasma is already available on the market (Noddeland, H. et al. Universal solvent/detergent-treated fresh frozen plasma (Uniplas—rationale and clinical properties. Thrombosis research 107 Suppl 1, S33-37 (2002); Solheim, B.G., Chetty, R. & Flesland, O. Indications for use and cost-effectiveness of pathogen-reduced ABO-universal plasma. Current opinion in hematology 15, 612-617 (2008)). However, this market is dominated worldwide by very few suppliers. These suppliers use artificially produced antigens or antigen fragments from erythrocytes for antibody adsorption. For example, WO 2007/100294 A1 describes a method which uses a binding molecule bound to a solid phase (matrix), optionally via a spacer molecule. The matrix materials described are polymers, polysaccharides or crosslinked polysaccharides, with the matrix being used in the form of beads or in the form of a filter. The binding molecule is selected from glycoproteins, saccharides and mucins. The antibody-depleted plasmas prepared in this way, so-called “universal plasmas”, are very expensive, and the methods of preparation are also time-consuming and material-intensive.

It is therefore an object of the invention to provide a method and a device, by means of which universal plasmas can be produced in a rapid, simple and cost-effective manner.

The object is achieved with a method for preparing blood plasma depleted in anti-A antibodies and/or anti-B antibodies, preferably free of anti-A antibodies and anti-B antibodies, comprising the steps of:
(a) providing blood plasma of blood group A, B or O or a mixture of two or more blood plasmas of blood group A, B and O;
(b) providing erythrocytes of blood group A and/or erythrocytes of blood group B and/or erythrocytes of blood group AB;
(c) combining the blood plasma according to (a) and the erythrocytes according to (b) to obtain a blood plasma depleted in anti-A antibodies and/or anti-B antibodies compared to the blood plasma according to (a) (depleted blood plasma), wherein the depleted blood plasma is preferably free of anti-A antibodies and/or anti-B antibody, and a cell sediment;
(d) removing the blood plasma depleted in anti-A antibodies and/or anti-B antibodies obtained according to (c), preferably the blood plasma free of anti-A antibodies and/or anti-B antibodies, from the cell sediment.

An advantage of this method is that a simple device, as will be described in detail below in relation to the fourth aspect of the invention, can be used. Said device makes it possible to add erythrocytes of blood group B, for example, to plasma of blood group A, for example, in a closed system. As a result, the anti-B antibody is adsorbed and the plasma can be given to the patient regardless of blood group. Erythrocytes of an erythrocyte concentrate of blood group B can be used for this purpose. From experience, these erythrocyte concentrates are rarely in demand, since the proportion of patients with blood group B is rather low at 9%.

An advantage of the method and the device is that this method can be used by any blood donation service at any time with the means available to it. Universal plasmas can thus be prepared as required. Another advantage is the manageable costs.

“Anti-A antibodies” means antibodies against blood group A, in particular against erythrocytes of blood group A, and “anti-B antibodies” means antibodies against blood group B, in particular against erythrocytes of blood group B. Anti-A antibodies preferably include at least anti-A immunoglobulin M (IgM) and/or anti-A immunoglobulin G (IgG), and anti-B antibodies preferably include at least anti-B immunoglobulin M (IgM) and/or anti-B immunoglobulin G (lgG). “Free of anti-A antibodies and/or anti-B antibodies” means that the blood plasma contains only low levels of anti-A antibodies and/or anti-B antibodies, with “low levels” meaning a maximum titer of 1:4 in relation to anti-A and/or anti-B antibodies. A person skilled in the art knows that antibody levels are not specified in absolute units, since there is no standard for this purpose. Moreover, the reaction of antibodies differs greatly depending on how high their binding strength is. This is why antibody titer levels, i.e., dilution levels, are specified, since they indicate, independently of the amount of antibodies and the binding strength thereof, the dilution in which the antibodies still trigger agglutination of erythrocytes. Dilution is carried out with isotonic saline solution (0.9% by weight aqueous NaCl solution). The titer specified is the dilution level at which the regular antibodies present in the plasma against erythrocytic A and B antigens only just react with always the same amount of test erythrocytes. The titer of an antibody that is specified is the reciprocal value of the last plasma dilution in which agglutinates are still visible. What is meant by 1:4 is that the plasma only had to be diluted 1:4 in order to still be able to visually identify agglutinates; in the next dilution (1:8), they are no longer visible. Dilution is carried out on the basis of volume, i.e., a titer of 1:4 means that one volume unit of plasma is mixed with three times the volume of diluent, in particular isotonic saline solution. The presence of agglutinates is determined using methods known to a person skilled in the art, such as the tube test or by means of a gel card (plastic card with microtiter columns, principle of size-exclusion chromatography), preference being given to the latter because of the higher sensitivity. Visual determination is carried out on the basis of possibly agglutinated erythrocytes which are lying on the gel surface after centrifugation (positive result) or of nonagglutinated erythrocytes which have migrated through the gel to the bottom of the tube (negative result).

“Cell sediment” means erythrocytes which have bound anti-A and/or anti-B antibodies, i.e., a reaction product composed of antibody and erythrocyte that is insoluble in blood plasma and that bears the relevant antigen and the antibodies bound thereto.

Blood plasma depleted in anti-A antibodies and/or anti-B antibodies, preferably blood plasma free of anti-A antibodies and anti-B antibodies, i.e., blood plasma which can be universally transfused to any recipient, is also referred to as “universal plasma”.

In one embodiment of the method, there is provided in (a) blood plasma of blood group A; there are provided in (b) erythrocytes of blood group B; and there is obtained in (c) a blood plasma depleted in anti-B antibodies. In an alternative embodiment of the method, there is provided in (a) blood plasma of blood group B; there are provided in (b) erythrocytes of blood group A; and there is obtained in (c) a blood plasma depleted in anti-A antibodies. In an alternative embodiment of the method,
there is provided in (a) blood plasma of blood group O; there are provided in (b) erythrocytes of blood group A, erythrocytes of blood group B and/or erythrocytes of blood group AB; and there is obtained in (c) a blood plasma depleted in anti-A antibodies and anti-B antibodies. In an alternative embodiment of the method, there is provided in (a) blood plasma of blood group A, B or O; there are provided in (b) erythrocytes of blood group AB; and there is obtained in (c) a blood plasma depleted in anti-A antibodies and anti-B antibodies.

Blood plasma is the component of blood that is liquid and virtually cell-free and that is obtained or obtainable by suitable measures, for example by plasmapheresis, sedimentation, centrifugation and/or size-exclusion filtration, for removing the blood cells (erythrocytes, thrombocytes and leukocytes) from a whole blood sample rendered incoagulable. As a result of the removal, blood plasma contains a very low level of residual cells, for example only leukocytes in a concentration of <0.1×10⁹/1, thrombocytes in a concentration of <50×10⁹/1, erythrocytes in a concentration of <6×10⁹/l (Richtlinie Hämotherapie [Directive on hemotherapy], complete amendment 2017), but—in contrast to blood serum-still contains all coagulation factors. The whole blood sample is rendered “incoagulable” by adding anticoagulants, preferably selected from the group consisting of sodium citrate, EDTA (ethylenediaminetetraacetate), heparin and mixtures of two or more of these substances. After at least the erythrocytes have been removed from the whole blood sample, the blood plasma obtained is optionally treated further. Preferably, the blood plasma is not enriched when the blood cells are removed from the whole blood, i.e., the blood plasma has the same concentration of anti-A antibodies and/or anti-B antibodies as the underlying whole blood to an extent of at least 90%.

In a preferred embodiment of the method, the blood plasma according to (a) is selected from the group consisting of fresh plasma, fresh frozen plasma, quarantine plasma, apheresis (plasmapheresis) plasma, irradiated plasma, filtered plasma, leukocyte-depleted plasma, pathogen-reduced, preferably pathogen-inactivated plasma and mixed forms of these plasma types. Mixed forms of these plasma types are known to a person skilled in the art and mean, for example, fresh apheresis plasma which is leucocyte-depleted and is frozen or was frozen. An example here is frozen and leukocyte-depleted fresh apheresis plasma which is registered, for example, under authorization/reg. No. (AMG76): PEI.H.01187.01.1. Pathogen reduction or pathogen inactivation of blood products serves the purpose of effectively reducing or inactivating most of the clinically relevant viruses, bacteria and protozoa. Pathogen reduction or pathogen inactivation also reduces or inactivates any leukocytes that may have remained in the blood plasma. Methods for pathogen reduction or pathogen inactivation are known to a person skilled in the art; examples include the Intercept method, the Mirasol method, the Theraflex MB method and the Theraflex UVC method.

In a preferred embodiment of the method, the blood plasma according to (a) contains one or more substances selected from the group consisting of (DNA, RNA)-intercalating substances, preferably amotosalen (UVA irradiation), riboflavin (UVB irradiation) and methylene blue (irradiation with visible light). Alternatively or additionally, the blood plasma according to (a) can be irradiated with UVC light or have been irradiated with UVC light without any of the aforementioned substances having been added. In a preferred embodiment of the method, the blood plasma according to (a) contains one or more anticoagulants, preferably selected from the group consisting of sodium citrate, EDTA, heparin and mixtures of two or more of these substances.

Erythrocytes, in particular erythrocyte concentrates (ECs), are obtained or obtainable from whole blood, as already described above in relation to blood plasma. Preferably, an erythrocyte concentrate is enriched when it is removed from the whole blood, i.e., the erythrocyte concentrate has a higher concentration of erythrocytes than the underlying whole blood; the erythrocyte concentrate specification according to Hämotherapie Richtlinie (Directive on hemotherapy) means a hematocrit in the range of 0.5-0.7 liter/liter (whole blood 0.3-0.5 liter/liter) and also hemoglobin >40 g/unit (unit: approx. 250 ml) (whole blood approx. 35 g/250 ml). The removal of the erythrocytes from the whole blood is usually carried out gravimetrically, preferably by centrifugation, with the pellet containing the erythrocytes being resuspended in additive solution, thereby yielding the erythrocyte concentrate.

Because most of the plasma in the erythrocyte concentrate has been replaced with additive solution, the plasma content of the erythrocyte concentrate is reduced to approx. 15 ml in approx. 300 ml. An “additive solution” is, as known to a person skilled in the art, a nutrient solution such as SAG-M (Sodium-Adenine-Glucose-Mannitol additive solution), PAGGS-M (phosphate, adenine, guanosine, glucose, sodium, mannitol) or ADSOL (adenine, dextrose, sodium, mannitol). Accordingly, 15 ml of erythrocyte concentrate still contain approx. 0.75 ml of plasma. If this erythrocyte concentrate of 15 ml is added to 1 liter of plasma for adsorption of the isoagglutinins, the amount of 0.75 ml of plasma is negligible.

In a preferred embodiment of the method, the erythrocytes of blood group A, erythrocytes of blood group B and/or erythrocytes of blood group AB according to (b) are provided in the form of an erythrocyte concentrate which has been obtained from whole blood, in particular by means of centrifugation and/or erythrocyte apheresis, wherein the erythrocyte concentrate preferably has a cell count in the range from 10¹⁰ to 10¹⁴ erythrocyte cells/liter, more preferably in the range from 10¹¹ to 10¹³ erythrocyte cells/liter, more preferably in the range from 1×10¹² to 1×10¹³ erythrocyte cells/liter, more preferably in the range from 3×10¹² to 9×10¹² erythrocyte cells/liter, more preferably in the range from 4×10¹² to 8.5×10¹² erythrocyte cells/liter, more preferably in the range from 5×10¹² to 8.2×10¹² erythrocyte cells/liter, more preferably in the range from 5.5×10¹² to 8×10¹² erythrocyte cells/liter, more preferably in the range from 5.96×10¹² to 7.7 6×10¹² erythrocyte cells/liter.

In a preferred embodiment of the method, the erythrocytes, preferably the erythrocyte concentrate, are selected from the group consisting of apheresis erythrocyte concentrate, irradiated erythrocyte concentrate, filtered erythrocyte concentrate, leukocyte-depleted erythrocyte concentrate, pathogen-reduced, preferably pathogen-inactivated erythrocyte concentrate, washed erythrocyte concentrate and mixed forms of these erythrocyte concentrates. Mixed forms of these erythrocyte concentrates are known to a person skilled in the art; one example is leukocyte-depleted and irradiated erythrocyte concentrate, as registered, for example, under authorization/reg. No. (AMG76): PEI.H.02806.01.1.

In a preferred embodiment of the method, the erythrocytes, preferably the erythrocyte concentrate, contain one or more anticoagulants, preferably selected from the group consisting of sodium citrate, EDTA, heparin, ACD-A solution and mixtures of two or more of these substances. In a preferred embodiment of the method, the erythrocytes, preferably the erythrocyte concentrate, contain one or more additives selected from the group consisting of citric acid, sodium citrate, glucose, dextrose, sodium dihydrogenphosphate, sodium monohydrogenphosphate, sodium phosphate, mannitol, adenine, guanosine, inosine, sodium chloride and mixtures of two or more of these additives.

Preferably, the erythrocytes according to (b), in particular erythrocyte concentrate, and blood plasma according to (a) are provided or combined according to (c) in an erythrocyte concentrate:blood plasma volume ratio in the range from 1:100 to 30:100, preferably in the range from 2:100 to 25:100, more preferably in the range from 3:100 to 20:100, more preferably in the range from 4:100 to 19:100. If erythrocyte suspensions or plasma are concentrated or diluted, the ratio between erythrocytes and plasma is adjusted in such a way that there are more antigens (A and/or B antigens) on the erythrocytes than corresponding soluble antibodies in the plasma.

In a preferred embodiment of the method, the erythrocytes of blood group B according to (b), in particular erythrocyte concentrate of blood group B, and blood plasma of blood group A according to (a) are provided or combined according to (c) in an erythrocyte concentrate:blood plasma volume ratio in the range from 2:100 to 6:100, preferably in the range from 3:100 to 5:100, more preferably in the range from 3.5:100 to 4.5:100, more preferably in the ratio of 4:100.

In a preferred embodiment of the method, erythrocytes of blood group A according to (b), in particular erythrocyte concentrate of blood group A, and blood plasma of blood group B according to (a) are provided or combined according to (c) in an erythrocyte concentrate:blood plasma volume ratio in the range from 2:100 to 6:100, preferably in the range from 3:100 to 5:100, more preferably in the range from 3.5:100 to 4.5:100, more preferably in the ratio of 4:100.

In a preferred embodiment of the method, erythrocytes of blood group A and/or erythrocytes of blood group B according to (b), preferably erythrocytes of blood group A and erythrocytes of blood group B, in particular erythrocyte concentrate of blood group A and erythrocyte concentrate of blood group B, and blood plasma of blood group O according to (a) are provided or combined according to (c) in an erythrocyte concentrate (A, B and/or AB):blood plasma volume ratio in the range from 10:100 to 30:100, preferably in the range from 12:100 to 25:100, more preferably in the range from 14:100 to 20:100, more preferably in the range from 17:100 to 19:100.

In a preferred embodiment of the method, erythrocytes of blood group AB according to (b), in particular erythrocyte concentrate of blood group AB, and blood plasma of blood group A, B or O according to (a) are provided or combined according to (c) in an erythrocyte concentrate (AB):blood plasma volume ratio in the range from 10:100 to 30:100, preferably in the range from 12:100 to 25:100, more preferably in the range from 14:100 to 20:100, more preferably in the range from 17:100 to 19:100.

In a preferred embodiment of the method, step (c) comprises:
(c.1) combining the blood plasma according to (a) and the erythrocytes according to (b) to obtain a mixture comprising blood plasma and erythrocytes;
(c.2) incubating the mixture obtained according to (c.1) for at least 10 minutes, preferably for a period of time in the range from 10 minutes to 48 hours, more preferably in the range from 30 minutes to 24 hours, more preferably in the range from 1 hour to 5 hours, more preferably in the range from 1.5 hours to 3 hours;
(c.3) separating blood plasma depleted in anti-A antibodies and/or anti-B antibodies, and cell sediment.

Preferably, (c.2) is carried out at a temperature in the range from 2 to 40° C., preferably in the range from 15 to 30° C., more preferably in the range from 20 to 25° C. Step (c.1) and/or step (c.2) is/are preferably carried out with active mixing, preferably by a method selected from the group consisting of swiveling, rotation, shaking, stirring and mixed forms of two or more of these methods, of blood plasma and erythrocytes. In the absence of active mixing, there is already sufficient reaction of anti-A antibodies and/or anti-B antibodies with the erythrocytes; in particular, the erythrocytes and the anti-A antibodies and/or anti-B antibodies already react with one another to a great extent, i.e., preferably to an extent of more than 50%, more preferably to an extent of 50%to 100%, more preferably to an extent of 60%to 100%, more preferably to an extent of 80%to 100%, more preferably to an extent of 90%to 100%, and so a blood plasma depleted in anti-A antibodies and/or anti-B antibodies and a reaction product composed of erythrocytes and anti-A antibodies and/or anti-B antibodies (cell sediment) are formed.

In a preferred embodiment of the method, the removal according to (d) is carried out by means of a method selected from the group consisting of centrifugation, decanting, filtration, sedimentation and mixtures of two or more of these methods.

In a preferred embodiment, the method further comprises:
testing the blood plasma depleted in anti-A antibodies and/or anti-B antibodies obtained according to (d) for agglutination, preferably by adding erythrocytes of blood groups A, B or AB and optionally anti-human globulin serum (Coombs serum) to a sample of the blood plasma depleted in anti-A antibodies and/or anti-B antibodies obtained according to (d), wherein an absence of an agglutinate, preferably determined visually, indicates that the blood plasma is free of anti-A antibodies and/or anti-B antibodies.

The method for preparing blood plasma depleted in anti-A antibodies and/or anti-B antibodies, preferably free of anti-A antibodies and anti-B antibodies, is preferably an in vitro method.

Preferably, the blood plasma or the whole blood sample comes from a mammal, more preferably from a human.

### Second Aspect—Blood Plasma Depleted In Anti-A Antibodies And/Or Anti-B Antibodies

In a second aspect, the invention relates to a blood plasma depleted in anti-A antibodies and/or anti-B antibodies, in particular anti-A antibody and/or anti-B antibody free blood plasma, obtained or obtainable by the method of the first aspect as described above.

### Third Aspect—Use

A third aspect of the invention relates to the use of a blood plasma depleted in anti-A antibodies and/or anti-B antibodies, in particular a blood plasma free of anti-A antibody and/or anti-B antibody, obtained or obtainable by the method of the first aspect described above or of a blood plasma depleted in anti-A antibodies and/or anti-B antibodies, in particular a blood plasma free of anti-A antibody and/or anti-B antibody, according to the second aspect for preparation of a transfusion solution, for preparation of a coagulation factor concentrate, for preparation of albumin, for preparation of immunoglobulin, for preparation of tissue adhesive, for preparation of a prothrombin complex concentrate (PPSB), for preparation of a cell culture, preferably a stem cell culture, wherein all the preparations mentioned are preferably carried out ex vivo or in vitro.

Furthermore, the invention relates to a blood plasma depleted in anti-A antibodies and/or anti-B antibodies, in particular a blood plasma free of anti-A antibody and/or anti-B antibody, obtained or obtainable by the method of the first aspect described above or a blood plasma depleted in anti-A antibodies and/or anti-B antibodies, in particular a blood plasma free of anti-A antibody and/or anti-B antibody, according to the second aspect for use in a therapeutic method or an in vivo diagnostic method or a surgical method, preferably for use in transfusion medicine for transfusion or before ABO-nonidentical organ transplantation or stem cell transplantation. In the case of ABO-nonidentical organ transplantation, anti-A antibodies and/or anti-B antibodies can damage a transplanted organ, in particular an organ selected from the group consisting of kidney, liver, heart and stem cell, if the transplanted organ comes from a donor of blood group A or blood group B and the recipient's blood contains anti-A antibodies and/or anti-B antibodies. Various methods are nowadays already used to remove anti-A antibodies and/or anti-B antibodies from the blood of a transplant recipient before organ transplantation.

Fourth Aspect-Device

According to a fourth aspect, the invention relates to a device for preparing blood plasma depleted in anti-A antibodies and/or anti-B antibodies, preferably free of anti-A antibodies and anti-B antibodies, comprising:
at least one collection container,
a connection device designed for connection of at least one first container, wherein the first container comprises blood plasma of blood group A, B or O or a mixture of two or more blood plasmas of blood group A, B and O, and at least one second container, wherein the second container comprises erythrocytes of blood group A, erythrocytes of blood group B and/or erythrocytes of blood group AB, to the collection container in such a way that the blood plasma of the first container is combinable with the erythrocytes of the second container in the collection container.

The device is suitable for depleting the blood plasma of anti-A antibodies and/or anti-B antibodies in such a way that the blood plasma contains essentially no anti-A antibodies and no anti-B antibodies. The expression “essentially no” refers to a technical realization in which anti-A antibodies and no anti-B antibodies are only present in technically unavoidable amounts in the final prepared blood plasma. “Free of anti-A antibodies and/or anti-B antibodies” means that the blood plasma contains only low levels of anti-A antibodies and/or anti-B antibodies, with “low levels” meaning a maximum titer of 1:4 in relation to anti-A and/or anti-B antibodies. Details in this regard have been described above in relation to the first aspect; they apply correspondingly to the device of the fourth aspect. As already described above in relation to the first aspect, the erythrocytes of the second container are preferably present as erythrocyte concentrates (ECs). Details in this regard have been described above in relation to the first aspect; they apply correspondingly to the device of the fourth aspect.

In a preferred embodiment of the device, the collection container is designed for mixing of the blood plasma of the first container with the erythrocytes of the second container.

In a preferred embodiment of the device, it comprises a mixing device, wherein the mixing device is designed for active mixing of the blood plasma of the first container with the erythrocytes of the second container in the collection container.

In a preferred embodiment of the device, the mixing device is designed for mixing of the blood plasma of the first container with the erythrocytes of the second container in the collection container by means of stirring, rotation, swiveling and/or movement of the collection container.

In a preferred embodiment of the device, the connection device comprises at least one channel, tube and/or flexible tube. The channel, tube and/or flexible tube is preferably made of a material authorized for medical purposes.

In a preferred embodiment of the device, it comprises multiple channels, tubes and/or flexible tubes, wherein at least some of the channels, tubes and/or flexible tubes are connectable or connected to one another.

In a preferred embodiment of the device, it further comprises a separation device, wherein the separation device is designed for removal of blood plasma depleted in anti-A antibodies and/or anti-B antibodies from cell sediment. The separation device is preferably at least one separation device selected from the group consisting of: separator, centrifuge, decanter, filter and settling tank.

In a preferred embodiment of the device, the connection device is designed for feeding of the blood plasma of the first container and the erythrocytes of the second container into the collection container by means of gravity or by means of a pump device.

In a preferred embodiment of the device, the connection device is designed for connection of the first container and the second container to the collection container by means of a screw connection, clamp connection, welded connection (sterile docking) or plug connection.

In a preferred embodiment of the device, the connection device is further designed for feeding of the depleted blood plasma to the first container and/or the second container.

In a preferred embodiment, the device further comprises a pump device, wherein the connection device is designed for feeding of the depleted blood plasma to the first container and/or the second container by means of the pump device. The pump device is preferably a manually operable pump device or an electric pump device.

In a preferred embodiment of the device, the first container, the second container and/or the collection container is at least partially made of plastic. In a preferred embodiment of the device, the first container, the second container and/or the collection container are each independently a bag or a bottle.

The present invention will be illustrated in greater detail by the following embodiments and combinations of embodiments arising from the relevant back-references and references. It should be noted here that in all cases which mention a range of embodiments, for example in the context of an expression such as “Method according to any of embodiments (1) to (4)”, each embodiment in this range is to be regarded as explicitly disclosed to a person skilled in the art, i.e., this expression is to be understood by a person skilled in the art as synonymous with “Method according to (each) one of embodiments (1), (2), (3) and (4)”. Furthermore, it is explicitly noted that the following set of embodiments is not the set of claims that defines the scope of protection; instead, it represents an appropriate structured part of the description that is directed to general and preferred aspects of the invention.

In one embodiment (1), the invention relates to a method for preparing blood plasma depleted in anti-A antibodies and/or anti-B antibodies, preferably free of anti-A antibodies and anti-B antibodies, comprising the steps of:
(a) providing blood plasma of blood group A, B or O or a mixture of two or more blood plasmas of blood group A, B and O;
(b) providing erythrocytes of blood group A and/or erythrocytes of blood group B and/or erythrocytes of blood group AB;
(c) combining the blood plasma according to (a) and the erythrocytes according to (b) to obtain a blood plasma depleted in anti-A antibodies and/or anti-B antibodies compared to the blood plasma according to (a) (depleted blood plasma), wherein the depleted blood plasma is preferably free of anti-A antibodies and/or anti-B antibody, and a cell sediment;
(d) removing the blood plasma depleted in anti-A antibodies and/or anti-B antibodies obtained according to (c), preferably the blood plasma free of anti-A antibodies and/or anti-B antibodies, from the cell sediment.

A preferred embodiment (2), which specificizes embodiment (1), relates to the method, wherein there is provided in (a) blood plasma of blood group A; there are provided in (b) erythrocytes of blood group B; and there is obtained in (c) a blood plasma depleted in anti-B antibodies; or wherein there is provided in (a) blood plasma of blood group B; there are provided in (b) erythrocytes of blood group A; and there is obtained in (c) a blood plasma depleted in anti-A antibodies, or wherein there is provided in (a) blood plasma of blood group O; there are provided in (b) erythrocytes of blood group A, erythrocytes of blood group B and/or erythrocytes of blood group AB; and there is obtained in (c) a blood plasma depleted in anti-A antibodies and anti-B antibodies, or wherein there is provided in (a) blood plasma of blood group A, B or O; there are provided in (b) erythrocytes of blood group AB; and there is obtained in (c) a blood plasma depleted in anti-A antibodies and anti-B antibodies.

A preferred embodiment (3), which specificizes embodiment (1) or (2), relates to the method, wherein the blood plasma according to (a) is selected from the group consisting of fresh plasma, fresh frozen plasma, quarantine plasma, apheresis (plasmapheresis) plasma, irradiated plasma, filtered plasma, leukocyte-depleted plasma, pathogen-reduced, preferably pathogen-inactivated plasma and mixed forms of these plasma types.

A preferred embodiment (4), which specificizes one of embodiments (1) to (3), relates to the method, wherein the blood plasma according to (a) contains one or more substances selected from the group consisting of (DNA, RNA)-intercalating substances, preferably amotosalen (UVA irradiation), riboflavin (UVB irradiation) and methylene blue (irradiation with visible light), and/or is irradiated with UVC light.

A preferred embodiment (5), which specificizes one of embodiments (1) to (4), relates to the method, wherein the blood plasma according to (a) contains one or more anticoagulants, preferably selected from the group consisting of sodium citrate, EDTA, heparin and mixtures of two or more of these substances.

A preferred embodiment (6), which specificizes one of embodiments (1) to (5), relates to the method, wherein the erythrocytes of blood group A, erythrocytes of blood group B and/or erythrocytes of blood group AB according to (b) are provided in the form of an erythrocyte concentrate which has been obtained from whole blood, in particular by means of centrifugation and/or erythrocyte apheresis, wherein the erythrocyte concentrate preferably has a cell count in the range from 10¹⁰ to 1014 erythrocyte cells/liter, more preferably in the range from 1011 to 1013 erythrocyte cells/liter, more preferably in the range from 1×10¹² to 1×10¹³ erythrocyte cells/liter, more preferably in the range from 3×10¹² to 9×10¹² erythrocyte cells/liter, more preferably in the range from 4×10¹² to 8.5×10¹²erythrocyte cells/liter, more preferably in the range from 5×10¹² to 8.2×10¹² erythrocyte cells/liter, more preferably in the range from 5.5×10¹² to 8×10¹² erythrocyte cells/liter, more preferably in the range from 5.96×10¹² to 7.7 6×10¹² erythrocyte cells/liter.

A preferred embodiment (7), which specificizes one of embodiments (1) to (6), relates to the method, wherein the erythrocytes, preferably the erythrocyte concentrate, are selected from the group consisting of apheresis erythrocyte concentrate, irradiated erythrocyte concentrate, filtered erythrocyte concentrate, leukocyte-depleted erythrocyte concentrate, pathogen-reduced, preferably pathogen-inactivated erythrocyte concentrate, washed erythrocyte concentrate and mixed forms of these erythrocyte concentrates.

A preferred embodiment (8), which specificizes one of embodiments (1) to (7), relates to the method, wherein the erythrocytes, preferably the erythrocyte concentrate, contain one or more anticoagulants, preferably selected from the group consisting of sodium citrate, EDTA, heparin, ACD-A solution and mixtures of two or more of these substances.

A preferred embodiment (9), which specificizes one of embodiments (1) to (8), relates to the method, wherein the erythrocytes, preferably the erythrocyte concentrate, contain one or more additives selected from the group consisting of citric acid, sodium citrate, glucose, dextrose, sodium dihydrogenphosphate, sodium monohydrogenphosphate, sodium phosphate, mannitol, adenine, guanosine, inosine, sodium chloride and mixtures of two or more of these additives.

A preferred embodiment (10), which specificizes one of embodiments (1) to (9), relates to the method, wherein erythrocytes according to (b), in particular erythrocyte concentrate, and blood plasma according to (a) are provided or combined according to (c) in an erythrocyte concentrate : blood plasma volume ratio in the range from 1:100 to 30:100, preferably in the range from 2:100 to 25:100, more preferably in the range from 3:100 to 20:100, more preferably in the range from 4:100 to 19:100.

A preferred embodiment (11), which specificizes one of embodiments (1) to (10), relates to the method, wherein erythrocytes of blood group B according to (b), in particular erythrocyte concentrate of blood group B, and blood plasma of blood group A according to (a) are provided or combined according to (c) in an erythrocyte concentrate:blood plasma volume ratio in the range from 2:100 to 6:100, preferably in the range from 3:100 to 5:100, more preferably in the range from 3.5:100 to 4.5:100, more preferably in the ratio of 4:100.

A preferred embodiment (12), which specificizes one of embodiments (1) to (11), relates to the method, wherein erythrocytes of blood group A according to (b), in particular erythrocyte concentrate of blood group A, and blood plasma of blood group B according to (a) are provided or combined according to (c) in an erythrocyte concentrate:blood plasma volume ratio in the range from 2:100 to 6:100, preferably in the range from 3:100 to 5:100, more preferably in the range from 3.5:100 to 4.5:100, more preferably in the ratio of 4:100.

A preferred embodiment (13), which specificizes one of embodiments (1) to (12), relates to the method, wherein erythrocytes of blood group A and/or erythrocytes of blood group B according to (b), preferably erythrocytes of blood group A and erythrocytes of blood group B, in particular erythrocyte concentrate of blood group A and erythrocyte concentrate of blood group B, and blood plasma of blood group O according to (a) are provided or combined according to (c) in an erythrocyte concentrate:blood plasma volume ratio in the range from 10:100 to 30:100, preferably in the range from 12:100 to 25:100, more preferably in the range from 14:100 to 20:100, more preferably in the range from 17:100 to 19:100.

A preferred embodiment (14), which specificizes one of embodiments (1) to (13), relates to the method, wherein erythrocytes of blood group AB according to (b), in particular erythrocyte concentrate of blood group AB, and blood plasma of blood group A, B or O according to (a) are provided or combined according to (c) in an erythrocyte concentrate (AB):blood plasma volume ratio in the range from 10:100 to 30:100, preferably in the range from 12:100 to 25:100, more preferably in the range from 14:100 to 20:100, more preferably in the range from 17:100 to 19 : 100.

A preferred embodiment (15), which specificizes one of embodiments (1) to (14), relates to the method, wherein step (c) comprises:
(c.1) combining the blood plasma according to (a) and the erythrocytes according to (b) to obtain a mixture comprising blood plasma and erythrocytes;
(c.2) incubating the mixture obtained according to (c.1) for at least 10 minutes, preferably for a period of time in the range from 10 minutes to 48 hours, more preferably in the range from 30 minutes to 24 hours, more preferably in the range from 1 hour to 5 hours, more preferably in the range from 1.5 hours to 3 hours;
(c.3) separating blood plasma depleted in anti-A antibodies and/or anti-B antibodies, and cell sediment.

A preferred embodiment (16), which specificizes embodiment (15), relates to the method, wherein (c.2) is carried out at a temperature in the range from 2 to 40° C., preferably in the range from 15 to 30° C., more preferably in the range from 20 to 25° C.

A preferred embodiment (17), which specificizes embodiment (15) or (16), relates to the method, wherein (c.1) and/or (c.2) is/are carried out with active mixing, preferably by a method selected from the group consisting of swiveling, rotation, shaking, stirring and mixed forms of two or more of these methods, of blood plasma and erythrocytes.

A preferred embodiment (18), which specificizes one of embodiments (1) to (17), relates to the method, wherein the removal according to (d) is carried out by means of a method selected from the group consisting of centrifugation, decanting, filtration, sedimentation and mixtures of two or more of these methods.

A preferred embodiment (19), which specificizes one of embodiments (1) to (18), relates to the method, which method is an in vitro method.

A preferred embodiment (20), which specificizes one of embodiments (1) to (19), relates to the method, wherein the blood plasma or the whole blood sample comes from a mammal, preferably from a human.

A preferred embodiment (21), which specificizes one of embodiments (1) to (20), relates to the method, further comprising:
testing the blood plasma depleted in anti-A antibodies and/or anti-B antibodies obtained according to (d) for agglutination, preferably by adding erythrocytes of blood groups A, B or AB and optionally anti-human globulin serum (Coombs serum) to a sample of the blood plasma depleted in anti-A antibodies and/or anti-B antibodies obtained according to (d), wherein an absence of an agglutinate, preferably determined visually, indicates that the blood plasma is free of anti-A antibodies and/or anti-B antibodies.

One embodiment (22) of the invention relates to a blood plasma depleted in anti-A antibodies and/or anti-B antibodies, in particular anti-A antibody and/or anti-B antibody free blood plasma, obtained or obtainable by the method according to one of embodiments (1) to (21).

One embodiment (23) of the invention relates to the use of a blood plasma depleted in anti-A antibodies and/or anti-B antibodies, in particular a blood plasma free of anti-A antibody and/or anti-B antibody, obtained or obtainable by the method according to one of embodiments (1) to (21) or of a blood plasma depleted in anti-A antibodies and/or anti-B antibodies, in particular a blood plasma free of anti-A antibody and/or anti-B antibody, according to embodiment (22) for preparation of a transfusion solution, for preparation of a coagulation factor concentrate, for preparation of albumin, for preparation of immunoglobulin, for preparation of tissue adhesive, for preparation of a prothrombin complex concentrate (PPSB), for preparation of a cell culture, preferably a stem cell culture.

One embodiment (24) of the invention relates to a blood plasma depleted in anti-A antibodies and/or anti-B antibodies, in particular blood plasma free of anti-A antibody and/or anti-B antibody, obtained or obtainable by the method according to one of embodiments (1) to (21) or blood plasma depleted in anti-A antibodies and/or anti-B antibodies, in particular blood plasma free of anti-A antibody and/or anti-B antibody, according to embodiment (22) for use in a therapeutic method or an in vivo diagnostic method or a surgical method, preferably for use in transfusion medicine for transfusion or before ABO-nonidentical organ transplantation or stem cell transplantation.

One embodiment (25) of the invention relates to a device for preparing blood plasma depleted in anti-A antibodies and/or anti-B antibodies, preferably free of anti-A antibodies and anti-B antibodies, comprising:
at least one collection container,
a connection device designed for connection of at least one first container, wherein the first container comprises blood plasma of blood group A, B or O or a mixture of two or more blood plasmas of blood group A, B and O, and at least one second container, wherein the second container comprises erythrocytes of blood group A, erythrocytes of blood group B and/or erythrocytes of blood group AB, to the collection container in such a way that the blood plasma of the first container is combinable with the erythrocytes of the second container in the collection container.

A preferred embodiment (26), which specificizes embodiment (25), relates to the device, wherein the collection container is designed for mixing of the blood plasma of the first container with the erythrocytes of the second container.

A preferred embodiment (27), which specificizes embodiment (25) or (26), relates to the device, further comprising a mixing device, wherein the mixing device is designed for active mixing of the blood plasma of the first container with the erythrocytes of the second container in the collection container.

A preferred embodiment (28), which specificizes embodiment (27), relates to the device, wherein the mixing device is designed for mixing of the blood plasma of the first container with the erythrocytes of the second container in the collection container by means of stirring, rotation, swiveling and/or movement of the collection container.

A preferred embodiment (29), which specificizes one of embodiments (25) to (28), relates to the device, wherein the connection device comprises at least one channel, tube and/or flexible tube.

A preferred embodiment (30), which specificizes embodiment (29), relates to the device, wherein the channel, tube and/or flexible tube is made of a material authorized for medical purposes.

A preferred embodiment (31), which specificizes embodiment (29) or (30), relates to the device, further comprising multiple channels, tubes and/or flexible tubes, wherein at least some of the channels, tubes and/or flexible tubes are connectable or connected to one another.

A preferred embodiment (32), which specificizes one of embodiments (25) or (31), relates to the device, further comprising a separation device, wherein the separation device is designed for removal of blood plasma depleted in anti-A antibodies and/or anti-B antibodies from cell sediment.

A preferred embodiment (33), which specificizes embodiment (32), relates to the device, wherein the separation device is at least one separation device selected from the group consisting of: separator, centrifuge, decanter, filter and settling tank.

A preferred embodiment (34), which specificizes one of embodiments (25) to (33), relates to the device, wherein the connection device is designed for feeding of the blood plasma of the first container and the erythrocytes of the second container into the collection container by means of gravity or by means of a pump device.

A preferred embodiment (35), which specificizes one of embodiments (25) to (34), relates to the device, wherein the connection device is designed for connection of the first container and the second container to the collection container by means of a screw connection, clamp connection, welded connection (sterile docking) or plug connection.

A preferred embodiment (36), which specificizes one of embodiments (25) to (35), relates to the device, wherein the connection device is further designed for feeding of the depleted blood plasma to the first container and/or the second container.

A preferred embodiment (37), which specificizes embodiment (36), relates to the device, further comprising a pump device, wherein the connection device is designed for feeding of the depleted blood plasma to the first container and/or the second container by means of the pump device.

A preferred embodiment (38), which specificizes embodiment (39), relates to the device, wherein the pump device is a manually operable pump device or an electric pumping device.

A preferred embodiment (39), which specificizes one of embodiments (25) to (38), relates to the device, wherein the first container, the second container and/or the collection container is at least partially made of plastic.

A preferred embodiment (40), which specificizes one of embodiments (25) to (39), relates to the device, wherein the first container, the second container and/or the collection container are each independently a bag or a bottle.

Furthermore, it is explicitly noted that the above set of embodiments is not the set of claims that defines the scope of protection; instead, it represents an appropriate structured part of the description that is directed to general and preferred aspects of the invention.

### DESCRIPTION OF THE FIGURES

FIG. 1 shows a graphical plot of the volume of erythrocyte concentrate of blood group B that is necessary for the complete depletion of anti-B antibodies in plasma of blood group A (n=15), with the respectively used volume of erythrocyte concentrate in milliliters on the Y-axis and the IgM or lgG isoagglutinin specificities on the X-axis.

FIG. 2 shows a graphic plot of the volume of erythrocyte concentrate of blood group B that was necessary for the complete depletion of anti-B IgG antibodies in plasma of blood group A at different incubation times (n=5), with the respectively used volume of erythrocyte concentrate in milliliters on the Y-axis and the incubation time in hours on the X-axis.

FIG. 3 shows one embodiment of the device for preparing blood plasma depleted in anti-A antibodies and/or anti-B antibodies.

FIG. 4 shows a photograph of one embodiment of the device comprising plasma-containing plasma bags, an erythrocyte concentrate-containing bag and empty collection bags.

The invention will be elucidated below on the basis of examples without being restricted thereto.

### EXAMPLES

Plasma unit: fresh frozen plasma from whole blood of blood group A
Erythrocyte concentrate: blood group B, cell count in the range from 5.96×10¹² to 7.76×10¹² erythrocyte cells/liter (standard deviation of 0.55×10¹² erythrocyte cells/liter, n=17)

### Example 1 Determination of the Volume of Erythrocyte Concentrate

First of all, the volume of erythrocyte concentrate of blood group B required to adsorb anti-B antibodies of one plasma unit of blood group A was determined.

Different volumes of erythrocyte concentrate of blood group B between zero ml (comparative sample) and 40 ml were each used to treat one plasma unit (300 ml) of blood group A for 1-5 hours at room temperature (20-24° C.). Thereafter, the agglutinates formed from erythrocytes and isoagglutinins were removed by means of centrifugation (e.g., 4000 g, 10 min) and subsequent separation by hand by pressing.

The antibody titer in relation to anti-B IgM and anti-B IgG was then determined as follows:
1. Preparation of a volume-based dilution series (1:1, 1:2, 1:4, 1:8, 1:16, 1:32) of the plasma, diluted using 0.9% by weight aqueous NaCl solution (isotonic saline solution)
2. Preparation of the test erythrocyte suspensions A1, B and O: 10 μl of erythrocyte sediment+1 ml of ID-Diluent 2 (Biorad)
3. 50 μl each of relevant erythrocyte suspension were added to the reaction chambers of a gel card (neutral and AH card for the determination of IgM or lgG antibodies; Biorad)
4. 50 μl each of plasma or plasma were added
5. Incubation at room temperature for 15 min
6. Centrifugation in a gel-card centrifuge (85 g, 10 min)
7. Visual assessment and documentation of the reaction

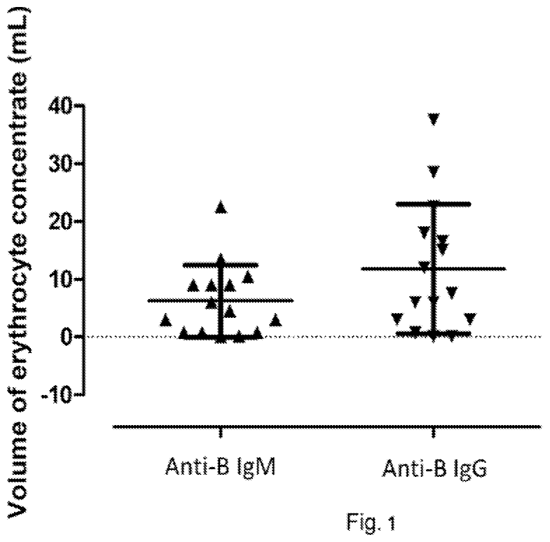

The results are shown graphically in FIG. 1. As can be seen from FIG. 1, an average of 12 ml of erythrocyte concentrate were required for removal of anti-B antibodies, in particular anti-B IgM and anti-B IgG, from plasma of blood group A.

### Example 2 Determination of the Incubation Time

Furthermore, the incubation time necessary for adsorption of the anti-B antibodies to the added erythrocytes was determined.

For this purpose, different volumes of erythrocyte concentrate of blood group B between zero ml (comparative sample) and 30 ml were each used to treat one plasma unit (300 ml) of blood group A. The samples were then left to stand at room temperature (20-24° C.) for a period of 1 to 5 hours.

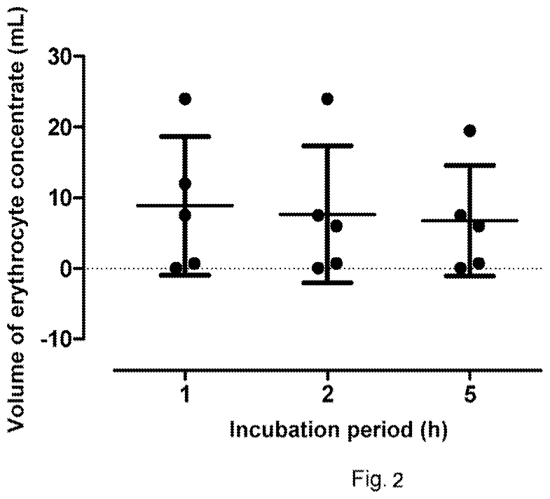

Thereafter, the antibody titer in relation to anti-B IgG was determined as specified in Example 1. The results are shown graphically in FIG. 2. On the basis of the results, 2 hours were determined as the optimal incubation time.

### Example 3 Configuration of a Device for Preparing Blood Plasma

A device for preparing blood plasma was developed, by means of which anti-B antibodies are removed or can be removed from plasma of blood group A.

The device comprises at least one collection container S, a connection device VV, a first container B1 and at least one second container B2. First and second containers B1, B2 are connected to the collection container S via the connection device VV in such a way that blood plasma of the first container B1 is combinable with the erythrocytes of the second container B2 in the collection container S. The collection container S is designed for mixing of the blood plasma of the first container B1 with the erythrocytes of the second container B2.

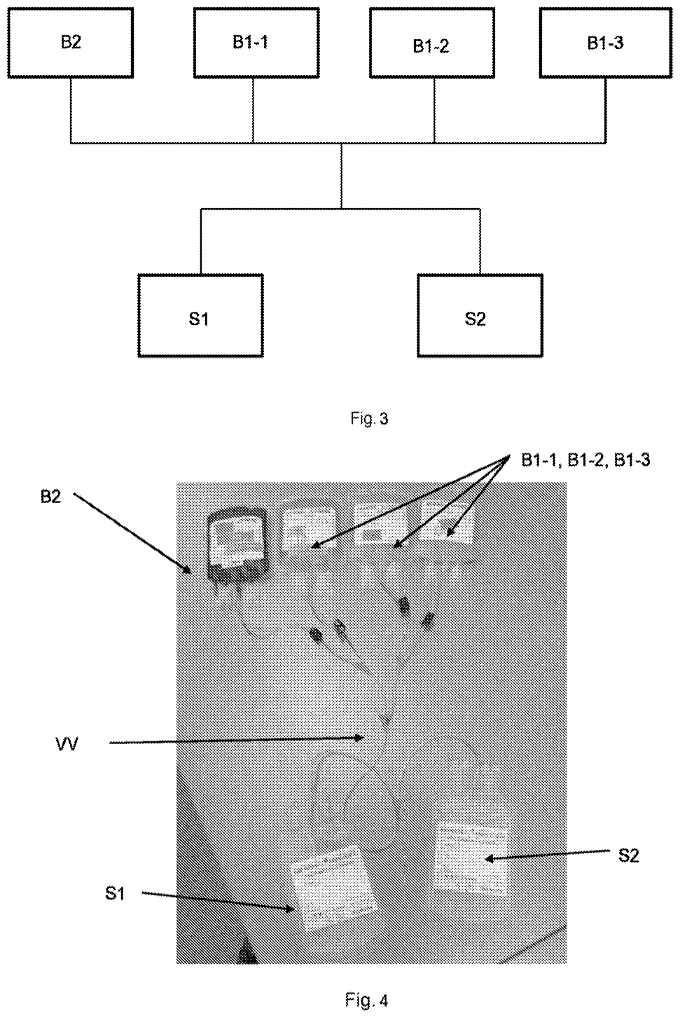

One embodiment of the device is shown schematically in FIG. 3, in which it was possible to carry out antibody depletion simultaneously for 3 plasma units. First container B1, second container B2 and collection container S were designed as bags, and the connection device VV was designed as a flexible tube system having a suitable number of connection points, 4 in this case. This device was also referred to as a blood bag system.

The two empty collection containers were two empty 500 ml bags S1, S2. They were welded via sterile docking to connection points of the flexible tube system and connected, via this, to 3 plasma bags B1-1, B1-2, B1-3, each containing 300 ml of blood plasma of blood group A, and one bag B2, containing 40 ml of erythrocyte concentrate of blood group B. FIG. 4 shows a photograph of the setup with plasma-containing plasma bags B1-1, B1-2, B1-3, erythrocyte-containing bag B2 and empty collection bags S1, S2.

### Example 4 Use of the device for Preparation of Antibody-Depleted Blood Plasma

The device in the embodiment shown in FIG. 3 was used. Plasma from the bags B1-1, B1-2, B1-3 and the erythrocyte concentrate from the bag B2 were transferred into the collection bags S1, S2 by gravity. Subsequently, after removal from the flexible tube system if necessary, the two filled collection bags S1 and S2 were incubated at room temperature (23° C.) for 2 hours and then centrifuged, and after redocking to the flexible tube system if necessary and after removal of the bag B2, the supernatant was pressed back into the original plasma bags B1-1, B1-2, B1-3, for example by application of an appropriate pressure to the collection bags S1 and S2. The plasma bags B1-1, B1-2, B1-3, containing the antibody-depleted plasma (universal plasma), were removed aseptically.

Thereafter, a check was carried out to determine whether antibodies were still present in the universal plasma prepared, i.e., the antibody titer in relation to anti-B IgM and anti-B IgG was determined as described in Example 1.

In all the tested dilution levels 1:1, 1:2, 1:4, 1:8, 1:16, 1:32, no agglutination could be detected by adding erythrocytes of blood group B to the universal plasma. This means that the anti-B antibodies were completely eliminated from the plasma.

The described device can be modified as follows. The device can comprise a mixing device designed for active mixing of the blood plasma of the first container B1 with the erythrocytes of the second container B2 in the collection container S. In principle, the active mixing can be carried out by means of stirring, rotation, swiveling and/or movement of the collection container S. As an alternative or in addition to the described flexible tubes, the connection device VV can comprise at least one channel or tube. The described flexible tubes and, if applicable, the optional channels or tubes are made of a material authorized for medical purposes. The device can comprise a separation device designed for removal of blood plasma depleted in anti-A antibodies and/or anti-B antibodies from cell sediment. The separation device can be at least one separator, centrifuge, decanter, filter and/or settling tank. The feeding of the blood plasma of the first container B1 and the erythrocytes of the second container B2 into the collection container S can be carried out by means of a pump device. The connection device can be further designed for feeding of the depleted blood plasma to the first container B1 and/or the second container B2. For example, the pump device can be used to feed the depleted blood plasma to the first container B1 and/or the second container B2. The pump device can be a manually operable pump device or an electric pump device. The connection device can be designed for connection of the first container B1 and the second container B2 to the collection container S by means of a screw connection, clamp connection or plug connection.

### Literature Cited

Noddeland, H. et al. Universal solvent/detergent-treated fresh frozen plasma (Uniplas --rationale and clinical properties. Thrombosis research 107 Suppl 1, S33-37 (2002).
Solheim, B.G., Chetty, R. & Flesland, O. Indications for use and cost-effectiveness of pathogen-reduced ABO-universal plasma. Current opinion in hematology 15, 612-617 (2008).
WO 2007/100294 A1

1. A method for preparing blood plasma depleted in anti-A antibodies and/or anti-B antibodies, comprising the steps of:
(a) providing blood plasma of blood group A, B or O or a mixture of two or more blood plasmas of blood group A, B and O;
(b) providing erythrocytes of blood group A and/or erythrocytes of blood group B and/or erythrocytes of blood group AB;
(c) combining the blood plasma according to (a) and the erythrocytes according to (b) to obtain a blood plasma depleted in anti-A antibodies and/or anti-B antibodies compared to the blood plasma according to (a), and a cell sediment, wherein the depleted blood plasma is free of anti-A antibodies and/or anti-B antibodies, wherein “free of anti-A antibodies and/or anti-B antibodies” means that the depleted blood plasma has a maximum titer of 1:4 in relation to anti-A and/or anti-B antibodies;
(d) removing the blood plasma depleted in anti-A antibodies and/or anti-B antibodies obtained according to (c) from the cell sediment.

2. The method as claimed in claim 1, wherein step (c) comprises:
(c.1) combining the blood plasma according to (a) and the erythrocytes according to (b) to obtain a mixture comprising blood plasma and erythrocytes;
(c.2) incubating the mixture obtained according to (c.1) for at least 10 minutes;
(c.3) separating blood plasma depleted in anti-A antibodies and/or anti-B antibodies, and cell sediment.

3. The method as claimed in claim 1, which method is an in vitro method.

4. The method as claimed in claim 1, wherein the blood plasma or the whole blood sample comes from a mammal.

5. A blood plasma depleted in anti-A antibodies and/or anti-B antibodies obtained or obtainable by the method as claimed in claim 1.

6. (canceled).

7. (canceled).

8. A device for preparing blood plasma depleted in anti-A antibodies and/or anti-B antibodies, comprising:
at least one collection container,
a connection device designed for connection of at least one first container, wherein the first container comprises blood plasma of blood group A, B or O or a mixture of two or more blood plasmas of blood group A, B and O, and at least one second container, wherein the second container comprises erythrocytes of blood group A, erythrocytes of blood group B and/or erythrocytes of blood group AB, to the collection container in such a way that the blood plasma of the first container is combinable with the erythrocytes in the collection container.

9. The device as claimed in claim 8, wherein the collection container is designed for mixing of the blood plasma of the first container with the erythrocytes of the second container.

10. The device as claimed in claim 8, wherein the connection device comprises at least one channel, tube and/or flexible tube.

11. The device as claimed in claim 8, wherein the connection device is designed for feeding of the blood plasma of the first container and the erythrocytes of the second container into the collection container by means of gravity or by means of a pump device.
